(19) United States
(12) Patent Application Publication
(10) Pub. No.: US 2017/0340802 A1
(43) Pub. Date: Nov. 30, 2017

(54) Method For Treating A Biological Fluid

(71) Applicant: United Arab Emirates University, Al Ain (AE)

(72) Inventors: Ali Hilal Alnaqbi, Al Ain (AE); Abdel Hamid Mourad, Al Ain (AE); Azza Abdulla Alshehhi, Al Ain (AE); Mariam Sultan Almarzooqi, Al Ain (AE); Rania Yaslam Aljaberi, Al Ain (AE); Sherina Matar Almuhairi, Al Ain (AE)

(21) Appl. No.: 15/490,594

(22) Filed: Apr. 18, 2017

Related U.S. Application Data
(60) Provisional application No. 62/341,905, filed on May 26, 2016.

(51) Int. Cl. A61M 1/36; B01J 20/20; A61K 35/14; C12N 5/071; B01J 20/30

(52) U.S. Cl. CPC A61M 1/3679; C12N 5/067; B01J 20/3085; B01J 20/3078; A61K 35/14; B01J 20/20; A61M 2205/3324; A61M 2205/3368

(57) ABSTRACT

A method for detoxifying a patient's blood by removing bilirubin from the patient's blood includes obtaining a batch of blood from the patient; controlling a pH of the blood so as to maintain the pH at approximately pH 7.4; controlling a temperature of the blood so as to maintain the temperature at approximately 37° C.; providing date pit-derived activated carbon; soaking the date pit-derived activated carbon within the blood for approximately 10-16 hours; and returning the detoxified blood to the patient.

### CROSS REFERENCE TO RELATED APPLICATION

This application claims the benefit of U.S. Provisional Application No. 62/341,905, filed on May 26, 2016, which is hereby incorporated by reference.

### FIELD OF INVENTION

This invention relates to a method for treating a biological fluid. More particularly, the invention relates to a method for treating blood in order to remove protein bound impurities, such as, for example, albumen bound toxins and/or bilirubin from the blood.

### BACKGROUND

In this specification, any reference to date pits shall be interpreted to mean the pits, pips or stones of a date bearing palm tree, such as, for example, but not limited to, Fard, Jabri, Lulu, Khunaizi and Khalas.

In this specification, any reference to the term “date pit-derived activated carbon” shall be interpreted to mean activated carbon derived from date pits. In this specification, any reference to the term “adsorption” shall be interpreted broadly to include also absorption and/or adsorption and visa versa, any reference to the term “absorption” shall be interpreted to include absorption and/or adsorption.

Liver failure and loss of function of liver cells below a critical level is a life threatening condition. Due to loss of function of the liver, protein bound toxins such as, for example, bilirubin, accumulate and adversely affect the biological and physiological mechanisms within the body. Bilirubin is an albumin bound toxin that acts as a standard clinical marker for liver failure.

Liver support devices assist patients until liver function recovers or until a liver transplant is performed. These liver support devices generally include adsorption units which remove albumen bound toxins from the blood. These adsorption units typically include activated carbon which acts as an adsorptive media in the liver support device.

Numerous methods are known for producing activated carbon from various kinds of raw materials such as, for example, lignin, sawdust, cherry stones and many other natural products, including date pits. US 2013/0089738 disclose methods for preparing activated carbon from date pits.

Activated carbon derived from date pits is known in water purification, for example, US 2013/0206688 discloses a process for reducing contaminants in a contaminated waste water stream which includes an adsorption column packed with activated carbon derived from date pits.

Commercially available activated carbon for use in medical applications is currently extremely costly. Accordingly, a need exists for activated carbon which is efficient at impurity removal and, at the same time, which is cost effective to produce and therefore substantially cheaper than commercially available activated carbon.

Furthermore, a need also exists for a commercially viable and profitable use for date pits which are a waste product of commercially produced pitted dates and which are currently either discarded or used for animal feed.

### SUMMARY OF INVENTION

According to a first aspect of the invention there is provided a method for treating a biological fluid obtained from a patient, for detoxifying the biological fluid, the method comprising:

providing date pit-derived activated carbon; and

contacting the biological fluid with the date pit-derived activated carbon.

The method may be an ex vivo method for treating the biological fluid.

The method for treating may, more particularly, be a method for detoxifying the biological fluid by removing protein bound impurities from the biological fluid.

In use, the contacting of the biological fluid with the date pit-derived activated carbon results in sorption of the protein bound impurities present in the biological fluid, by the date pit-derived activated carbon.

The contacting of the biological fluid with the date pit-derived activated carbon may comprise, more particularly, soaking the date pit-derived activated carbon within the biological fluid. More specifically, the date pit-derived activated carbon may be soaked within the biological fluid for a period of time of approximately 10-16 hours.

Providing date pit-derived activated carbon may include providing date pits and physically and chemically activating the date pits so as to obtain the date pit-derived activated carbon. The physical activation may include carbonizing the date pits in an inert atmosphere. The physical activation may further include exposing the carbonized date pits at an elevated temperature to at least one of oxidizing gasses and steam. Chemical activation may include exposing the carbonized date pits to dehydrating chemicals. More specifically, providing date pit-derived activated carbon may include grinding date pits to obtain date pit granules; carbonizing the date pit granules in a furnace having an initial temperature of 5° C.; flowing nitrogen gas over the granules for ten minutes; increasing the temperature at a rate of 5° C./min up to 600° C.; maintaining the temperature for about an hour; allowing the carbonized granules to cool to room temperature. The method may further include physical activation of the carbonized granules at 900° C. for approximately two hours in the furnace under the flow of carbon dioxide.

The method may include grinding the date pit-derived activated carbon so as to obtain granules of the date pit-derived activated carbon. In a particular embodiment, the method may include sieving the date pit-derived activated carbon through a sieve having apertures in the range of from about 500 μm to 600 μm. In another embodiment, the method may include grinding the date pit-derived activated carbon so as to obtain nano material in the form of nanoparticles of the date pit-derived activated carbon. The nanoparticles of the date pit-derived activated carbon may be of a particle size of about 60-200 nano meter.

The method may further include controlling a pH of the biological fluid. More specifically, the pH of the biological fluid may be controlled so as to maintain the pH at approximately pH 6.9-7.9, preferably pH 7.4. As such, the method may, more particularly, include providing a sensor for measuring a pH of the biological fluid.

The method may further include controlling a temperature of the biological fluid. More specifically, the temperature of the biological fluid may be controlled so as to maintain the temperature at approximately 36.9-37.9° C. preferably approximately 37° C. As such, the method may include providing a sensor for sensing a temperature of the biological fluid. Furthermore, the method may include providing a heater for heating the biological fluid.

The method may further include providing a vessel within which the date pit-derived activated carbon is located. The method may further include providing a tank for containing the biological fluid and displacing the biological fluid between the tank and the vessel, so as to soak the date pit-derived activated carbon within the biological fluid. As such, the method may further include providing fluid displacing means for displacing the biological fluid between the tank and the vessel. The method may include providing a heating element within the vessel. As such, the temperature of the biological fluid within the vessel may be increased by activation of the heating element.

The patient, from whom the biological fluid was obtained, may be in need of treatment. As such, the method may include obtaining the biological fluid from the patient. More particularly, the method may include treating batches of the biological fluid, whereby a volume of the biological fluid is obtained from the patient and thereafter, the treated biological fluid is returned to the patient. The biological fluid may be in the form of one of blood and plasma.

The method may be, more particularly, for the removal of protein bound impurities from the patient's blood. The protein bound impurities removed from the blood may, more particularly, be in the form of albumin bound toxins. The protein bound impurities removed from the blood may be in the form of bilirubin.

The method may be used to treat one of blood and plasma from a patient having one of liver disease; loss of liver function; and liver failure. Furthermore, the method may be used to treat one of blood and plasma of a patient undergoing treatment by one of a liver support device and a dialysis device. The patient may be one of a mammal and a human.

The date pit-derived activated carbon may be obtained by a process for producing the date pit-derived activated carbon from date pits, as described and defined hereinbelow.

According to a second aspect of the invention there is provided date pit-derived activated carbon for use in a method for treating a biological fluid for removing protein bound impurities from the biological fluid. The date pit-derived activated carbon may be derived from date pits, in accordance with a method of producing date pit-derived activated carbon as described in more detail hereinbelow.

According to a third aspect of the invention there is provided an apparatus for treating biological fluid, the apparatus including date pit-derived activated carbon. The date pit-derived activated carbon may be, as described and defined hereinabove, in accordance with the first aspect of the invention.

The invention extends also to the use of date pit-derived activated carbon in the manufacture of a pharmaceutical composition and/or permeable membranes for treating biological fluid, for removing protein bound impurities from the biological fluid.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Further features of the invention are described hereinafter by way of a non-limiting example of the invention, with reference to and as illustrated in the accompanying diagrammatic drawings. In the drawings:

FIG. 1A shows a Scanning Electron Microscope (SEM) image of raw date pits;

FIG. 1B shows another SEM image of raw date pits;

FIG. 1C shows yet another SEM image of raw date pits;

FIG. 2A shows a SEM image of date pit-derived activated carbon formed after activation of the raw date pits of FIGS. 1A to 1C;

FIG. 2B shows another SEM image of date pit-derived activated carbon formed after activation of the raw date pits of FIGS. 1A to 1C;

FIG. 2C shows a different SEM image of date pit-derived activated carbon formed after activation of the raw date pits of FIGS. 1A to 1C;

FIG. 3A shows a SEM image of bilirubin adsorption over the date pit-derived activated carbon of FIGS. 2A to 2C;

FIG. 3B shows another SEM image of bilirubin adsorption over the date pit-derived activated carbon of FIGS. 2A to 2C;

FIG. 3C shows a different SEM image of bilirubin adsorption over the date pit-derived activated carbon of FIGS. 2A to 2C;

FIG. 4 shows a graph showing a fraction of bilirubin left after each interval of time for 416 nm in albumin free solution;

FIG. 5 shows a graph showing a fraction of bilirubin left after each interval of time for 350 nm in albumin free solution;

FIG. 6 shows a graph showing a fraction of bilirubin left after each interval of time for 416 nm in albumin bound bilirubin containing solution;

FIG. 7 shows a graph showing albumin left after each interval of time for 350 nm in albumin bound bilirubin solution;

FIG. 8 shows a perspective view of an apparatus, in accordance with the third aspect of the invention, for practicing a method in accordance with the first aspect of the invention;

FIG. 9 shows a table showing physio-chemical characteristics of activated carbon samples derived from date pits, in accordance with the invention, as well as activated carbon derived from Jojoba and Algae (Scenedesmus sp);

FIG. 10A shows morphology of viable THLE2 cells (control) by Hematoxylin and Eosin staining using a Bright Field microscope;

FIG. 10B shows morphology of viable THLE2 cells treated with Nano DP-AC by Hematoxylin and Eosin staining using a Bright Field microscope;

FIG. 11 shows a graph showing viability by MTT assay of THLE2 cells treated with Nano DP-AC for 1st, 2nd and 3rd hours respectively;

FIG. 12 shows a graph showing time dependence of remaining bilirubin in a solution using date pit-derived activated carbon in accordance with the invention, Jojoba-derived activated carbon (Jojoba-AC), and microalgae-derived activated carbon (microalgae-AC) at 37° C., pH 7.4 and initial bilirubin concentration of 30 μM;

FIG. 13 shows a graph showing time dependence of remaining albumen in a solution using date pit-derived activated carbon in accordance with the invention, Jojoba-derived activated carbon (Jojoba-AC), and microalgae-derived activated carbon (microalgae-AC) at 37° C., pH 7.4 and initial bilirubin concentration of 30 μM;

FIG. 14 shows the effect of the amount of date pit-derived activated carbon in accordance with the invention, used on the time dependence of remaining bilirubin concentration at 37° C., pH=7.4 and initial bilirubin concentration of 30 μM;

FIG. 15A shows a scanning electron microscope (SEM) image of raw date pit (×1,500 magnification);

FIG. 15B shows a scanning electron microscope (SEM) image of date pit derived activated carbon in accordance with the invention, (×1,500 magnification);

FIG. 16 shows results of a graph showing FTIR spectroscopy for (a) date pits, (b) date pit-derived activated carbon (DP-AC) in accordance with the invention;

FIG. 17 shows a graph showing Differential Scanning Calorimetry for date pit-derived activated carbon (DP-AC) in accordance with the invention (heating rate of 10° C. with a nitrogen flow rate of 50 ml/min);

FIG. 18 show a graph showing the results of Thermogravimetric and Derivative thermogravimetric analysis for date pit derived activated carbon (DP-AC) in accordance with the invention;

FIG. 19 shows a graph showing the effect of initial bilirubin concentration on the final equilibrium concentration using 0.8 g DP-AC in accordance with the invention, Jojoba-AC, microalgae-AC at 37° C. and pH 7.4;

FIG. 20 show a table showing isotherms parameters of the adsorption of bilirubin on DP-AC in accordance with the invention, Jojoba-AC, and microalgae-AC at 37° C. and pH 7.4;

FIG. 21 shows a table showing bilirubin adsorption capacities of different adsorbents;

FIG. 22 shows log [1−(qₜ/qₒ)] vs. t plot for bilirubin adsorption on DP-AC in accordance with the invention, Jojoba-AC and microalgae-AC at 37° C. and pH 7.4;

FIG. 23 shows a table showing fitted kinetics parameters of the adsorption of bilirubin on DP-AC in accordance with the invention, Jojoba-AC, and microalgae-AC at 37° C. and pH 7.4;

FIG. 24 shows

$$\frac{1}{{q}_{o} - q} - \frac{1}{{q}_{o}}$$

vs t plot for bilirubin adsorption on DP-AC in accordance with the invention, Jojoba-AC and microalgae-AC at 37° C. and pH 7.4; and

FIG. 25 shows qₜ vs t^{1/2} plot for bilirubin adsorption on DP-AC in accordance with the invention, Jojoba-AC and microalgae-AC at 37° C. and pH 7.4.

### DETAILED DESCRIPTION

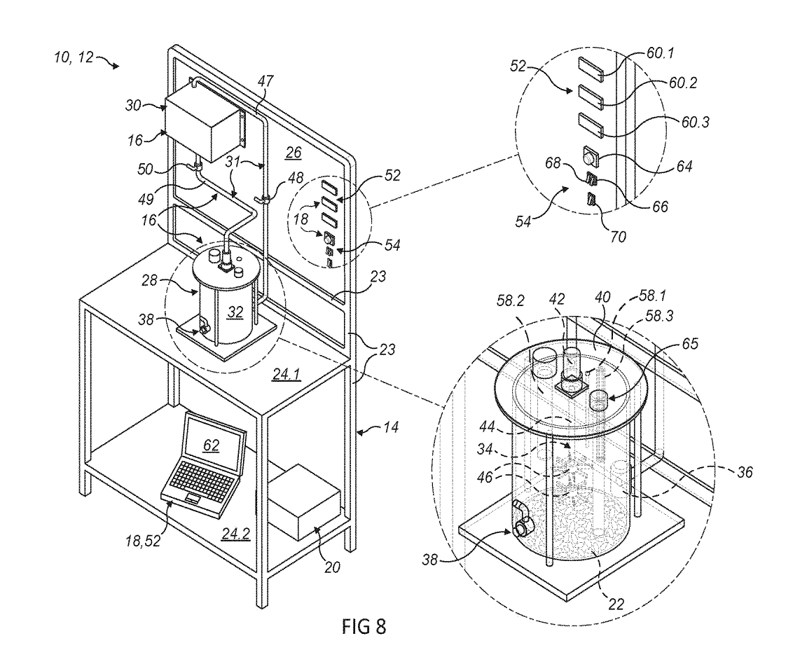

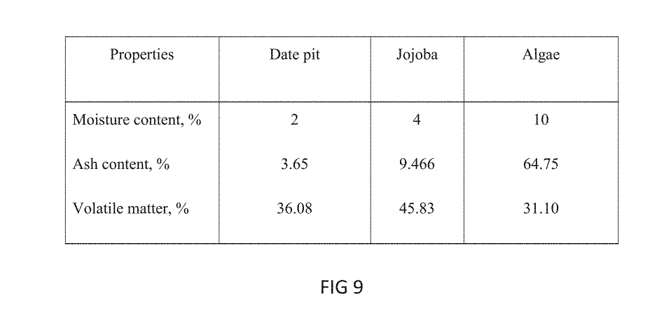

FIG. 8 of the drawings shows an apparatus, in accordance with the third aspect of the invention, in the form of a liver support device 10 which is configured for carrying out an ex vivo method 12, in accordance with the first aspect of the invention, for treating a biological fluid in the form of blood. The apparatus 10 is, more particularly, configured for us in the method 12 for detoxifying blood by removing protein bound impurities, such as, for example, albumen bound toxins and/or bilirubin from the blood.

The apparatus 10 includes broadly a support stand 14; a blood holding and transport system 16; a signaling and control system 18; a power supply system 20 and date pit-derived activated carbon 22, the purpose of which will be explained in more detail hereinbelow.

The support stand 14 is configured for supporting the components of the liver support device 10, including the blood holding and transport system 16; the signaling and control system 18; and the power supply system 20. The support stand 14 includes a framework structure 23 of metal tubing for supporting shelves 24.1, 24.2 and a vertical support panel 26. The stand 14 renders the liver support device 10 portable and securely holds the components of the liver support device 10 at ergonomic and easily accessible positions.

The blood holding and transport system 16 includes a vessel assembly 28; a tank 30 for containing blood; and a conduit system 31 for carrying blood between the vessel assembly 28 and the tank 30.

The vessel assembly 28 includes an open topped vessel 32 for containing blood; a stirrer assembly 34; a pump assembly 36; an outlet assembly 38; and a support and closure plate 40 for closing the open top of the vessel 32 and for supporting various components of the liver support device 10, as will be explained in more detail hereinbelow.

The vessel 32 is of polymethyl methacrylate, also known as acrylic glass, and is configured to hold approximately 2 litres of blood.

The stirrer assembly 34 includes a stainless steel stirrer shaft 44; a stirrer motor 42 arranged for causing rotational displacement of a stirrer shaft 44 and a pair of stainless steel stirrer formations 46 mounted to lower end regions of the stirrer shaft 44 and disposed at lower regions of the vessel 32, for stirring the blood located within the vessel 32.

The pump assembly 36 is mounted within the vessel 32 at a lower end region of the vessel and includes a pump for pumping blood between the vessel and the tank 30, as will be explained in more detail hereinbelow.

The outlet assembly 38 is configured for releasing treated blood from the blood holding and transport system 16. The outlet assembly 38 is located at a lower region of the vessel 32 and includes an outlet pipe and a valve for controlling opening and closing of the outlet pipe, for controlling the release of treated blood from the vessel 32, as will be explained in more detail hereinbelow.

The support and closure plate 40 has a disc-like configuration and includes a central aperture extending therethrough, for receiving and securely holding the stirrer motor 42, as illustrated in FIG. 8 of the drawings. The support and closure plate 40 further includes a number of other apertures extending therethrough for receiving and holding various other components of the liver support device 10, as shown in FIG. 8 of the drawings and as will be explained in more detail below.

The tank 30 is fixedly secured to the vertical support panel 26 at a position located above the vessel assembly 28. The tank 30 is configured to contain approximately two litres of blood.

The conduit system 31 includes a vessel outlet pipe 47, an outlet valve 48, a vessel inlet pipe 49 and an inlet valve 50. The vessel outlet pipe 47 is connected between a lower end of the vessel 32 and an upper end of the tank 30 for carrying blood from the vessel 32 to the tank 30 when the pump of the pump assembly 36 is activated, as will be explained in more detail hereinbelow. The vessel inlet pipe 49 is connected between a lower end of the tank 30 and an upper end of the vessel 32, as shown in FIG. 8 of the drawings, for carrying blood from the tank 30 to the vessel 32, under action of gravity, when the inlet valve 50 is open, as will be explained in more detail hereinbelow.

The signaling and control system 18 includes a signaling system 52 and a control system 54.

The signaling system 52 includes three sensors 58; three parameter displays 60 and a computer 62 connected between the sensors 58 and the parameter displays 60, for receiving signals from the sensors 58 and for sending a signal to each of the parameter displays 60 for displaying a parameter value on each of the parameter displays 60, as will be explained in more detail hereinbelow.

The sensors 58 include a partial pressure sensor 58.1 for checking the oxygen availability to ensure that the availability of oxygen is not less than 2 mmHg, a pH sensor 58.2 for sensing pH of the blood in the vessel 32, and a temperature sensor 58.3 for sensing the temperature of the blood in the vessel. The parameter displays 60 include a partial pressure display 60.1; a pH display 60.2; a temperature display 60.3.

The control system 54 includes a motor speed controller 64 for controlling a speed of the stirrer motor 42; a heater element 65 mounted within one of the apertures of the support and closure plate 40 of the vessel assembly 28; a heater switch 66 for controlling activation of the heater element 65; a pump switch 68 for controlling activation of the pump of the pump assembly 36; and a stirrer motor switch 70 for controlling activation of the stirrer motor 42.

The power supply system 20 is electrically connected to the signaling and control system 18, the pump assembly 36 and to the stirrer motor 42 for supplying electrical energy thereto, in use, for energizing the signaling and control system 18, the pump assembly 36 and to the stirrer motor 42.

The date pit-derived activated carbon 22 is located within the vessel 32. More particularly, the date pit-derived activated carbon 22 is randomly packed within the vessel 32 and is supported upon an upper surface of a base of the vessel 32, as illustrated in FIG. 8 of the drawings.

Referring to FIG. 8 of the drawings, in use, the apparatus 10 is used to carry out the method 12 for treating the blood. The method 12 for treating the blood will be described in more detail hereinbelow, with reference to the use of the apparatus 10.

In accordance with the method 12, blood is obtained from a human patient undergoing treatment with a dialysis device or a liver support device, as the patient has liver disease and is experiencing loss of liver function. The blood is obtained using known conventional clinical methods for obtaining a batch of blood from a patient.

In use, the patient's blood is transferred to the tank 30 of the apparatus 10. The inlet valve 50 is opened and the blood flows under the action of gravity from the tank 30 through the vessel inlet pipe 49 and into the vessel 32.

The date pit-derived activated carbon 22 is soaked in the blood for approximately 16 hours, or less if desired. In use, the contact between the blood and the date pit-derived activated carbon 22 results in sorption of the protein bound impurities present in the blood, by the date pit-derived activated carbon 22.

In use, the stirrer motor 42 is actuated, causing rotation of the stirrer formations 46 for stirring the blood within the vessel 32 so at to reduce and/or prevent coagulation and/or clotting of the blood within the vessel 32.

The signaling and control system 18 is used to monitor and regulate the pressure, pH and/or temperature of the blood. More specifically, the heater element 65 is actuated via the heater switch 66 for heating the blood within the vessel 32. To reduce the temperature of the blood, in use, the heater switch 66 is open, so as to cut electrical power to the heater element 65 and the pump switch 68 is closed for actuating the pump of the pump assembly 36 for pumping blood between the vessel 32 and the tank 30, so as to displace blood from the vessel 32 to the tank 30. In use, when the inlet valve 50 is open and when the pump of the pump assembly 36 is actuated, blood is thus caused to circulate, as blood flows under the action of gravity, from the tank 30 to the vessel 32 and is again pumped by the pump of the pump assembly 36 from the vessel 32 to the tank 30. This circulation of the blood clause a reduction in the temperature of the blood. As such, the temperature of the blood is maintained at a constant temperature of 37° C.

The pH sensor 58.2 senses the pH of the blood and provides a pH value on the pH display 60.2 for monitoring the pH of the blood, to enable a user of the apparatus 10 to ensure that the pH of the blood is maintain a pH of approximately 7.4.

It will be understood that the method 12, in accordance with the invention, may be practiced using another apparatus (not shown) which is different from the apparatus 12 described hereinabove.

The date pit-derived activated carbon 22 is obtained by an activation process for producing the date pit-derived activated carbon 22 from date pits.

The activation process includes physical activation and chemical activation. Physical activation is a two step process. During physical activation the carbonization of the date pit precursor occurs in an inert atmosphere. The resulting char is activated by suitable oxidizing gases such as carbon dioxide, steam or their mixtures at an elevated temperature, as will be explained in more detail hereinbelow. This reaction results in removal of carbon atoms and in the process simultaneously produces a wide range of pores, resulting in porous activated carbon. Physical activation is favorable for commercial production because of its simplicity of process and yields desirable physical characteristics such as the good physical strength and porosity. While in chemical activation process the precursors are impregnated with a variety of dehydrating chemicals and carbonized at desired conditions generally in a single step.

More particularly, the activation process begins with grinding date pits so as to obtain date pit granules. The date pit granules are washed several times with water and dried. The date pit granules are then used as a precursor for the preparation of the date pit-derived activated carbon 22. Carbonization of the granules is performed in a tube furnace followed by activation of the carbonaceous material. During carbonization, initially flow of a nitrogen gas over the granules is carried out for 10 minutes and then the temperature is gradually increased at a rate of 5° C./min up to 600° C. This temperature is then maintained for about four hours. The carbonized granules are then allowed to cool. After cooling to room temperature the carbonized granules are removed and weighed separately, followed by activation of the carbonaceous material at 900° C. for approximately two hours in the furnace under the flow of carbon dioxide.

The inventors have surprisingly found that the date pit-derived activated carbon 22 is effective in removing bilirubin and/or albumen from a mixture and/or solution containing the bilirubin and/or albumen. More specifically, the invention will be further understood with reference to the following non-limiting example which is provided for exemplification purposes. The particular example, materials, amounts and procedures are not intended to limit the scope of the invention.

### Example

### Materials and Methods

Albumin and bilirubin were purchased from Sigma-Aldrich Company Ltd. (of The Old Brickyard, New Road, Gillingham, Dorset, SP8 4XT, United Kingdom) and used as received. All the chemicals used were of analytical grade. All experiments were conducted in a dark room to avoid photo degradation and stability of the solutions was tested for period of 24 hours by running control experiments without the activated carbon. The bilirubin was weighed and mixed with 0.1 M NaOH and after dissolving with NaOH, it was added in a solution of phosphate buffer saline (PBS) at pH 7.4. The final pH of the solution is maintained at 7.4. The experiments were conducted with and without albumin. The bilirubin (PBS) and albumin bound bilirubin (PBS) is serially diluted for different concentrations. To these concentrations activated carbon of different amounts were added. Then it is kept in shaker which is maintained at a temperature of 37° c. and constant speed. The analysis of the solutions was carried spectrophotometrically by a UV-Visible spectrophotometer. Readings were measured initially from zero to four hours and final reading for overnight sample. Total bilirubin concentration was evaluated by calibrating at the isobestic point (λ=416 nm) and albumin at 350 nm. After 16 hours, the solutions of different concentrations were taken and centrifuged at 4500 rpm and the supernatant filtered and analyzed.

### Statistical Analysis

All the values are given as the mean of the three samples unless otherwise stated. To present error bars standard deviation is calculated.

### Scanning Electron Microscopy (SEM)

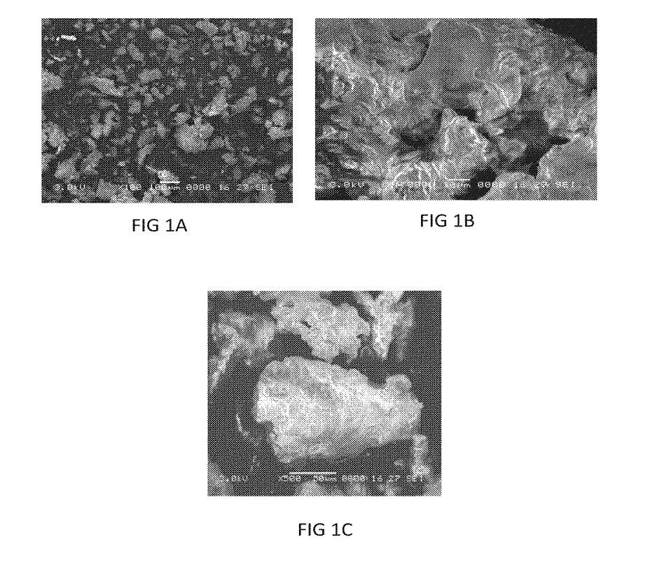

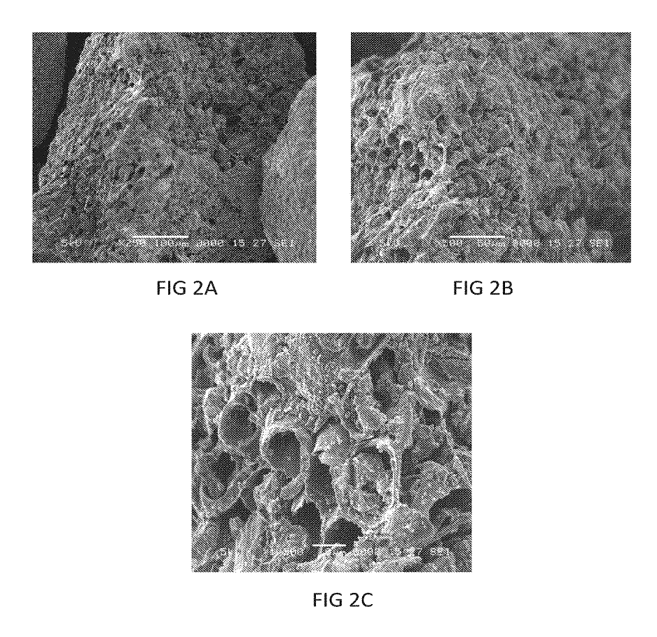

Changes in the surface morphology of date pit activated carbon before and after activation were examined by scanning electron microscopy which showed the pore size of date-pit derived activated carbon 22. FIGS. 1A, 1B, and 1C show SEM images of raw date pits before activation. FIGS. 2A, 2B and 2C show SEM images of date pit-derived activated carbon 22 formed after activation of the raw date pits of FIGS. 1A to 1C.

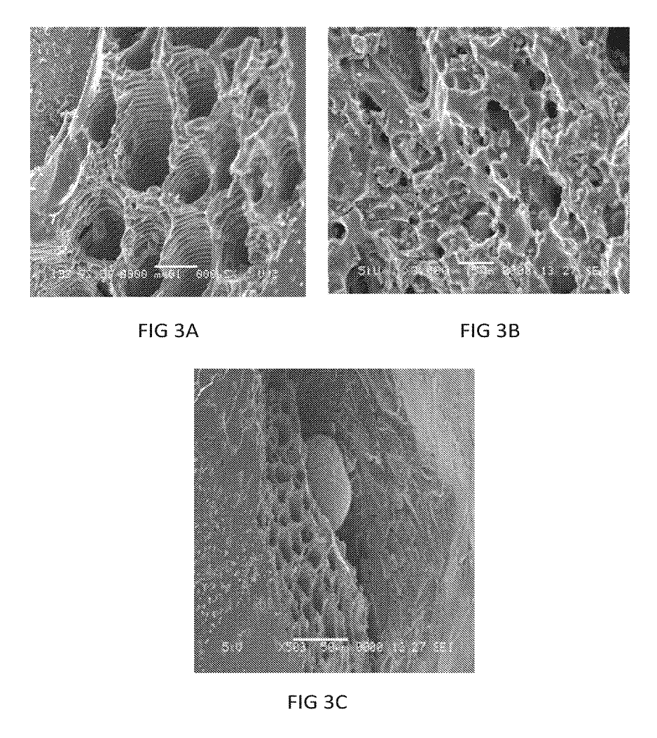

FIGS. 3A, 3B and 3C show the images of bilirubin adsorbed over the date pit-derived activated carbon 22 of FIGS. 2A to 2C.

### Results and Discussion

Batch experiments with albumin bonded bilirubin were carried out in order to evaluate bilirubin adsorption onto the date pit-derived activated carbon 22. The adsorption experiments were studied at pH 7.4 and at a temperature of 37° C.

Various amounts of the date pit-derived activated carbon 22 were added in different concentrations to show the adsorption of bilirubin at wavelengths of 416 nm and 350 nm. From spectrophotometric readings, graphs were plotted which show a reduction in bilirubin after each interval of time. The results show that bilirubin adsorbed at its maximum after 16 hours of incubation. Maximum adsorption of bilirubin was observed at the lower dilutions and higher amount of the date pit-derived activated carbon 22. Further experiments at different pH and temperature may still be necessary to determine adsorption at various other pH and temperature.

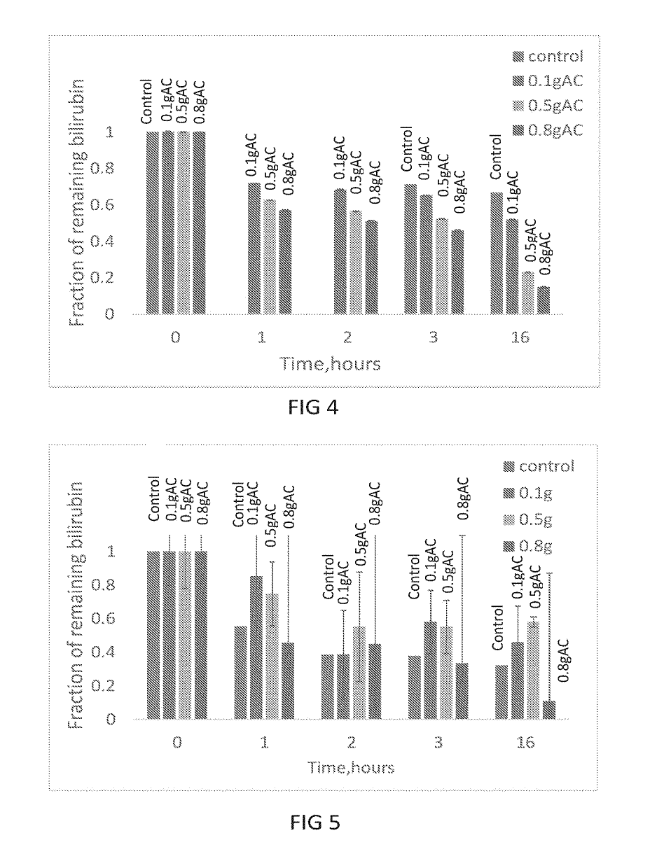

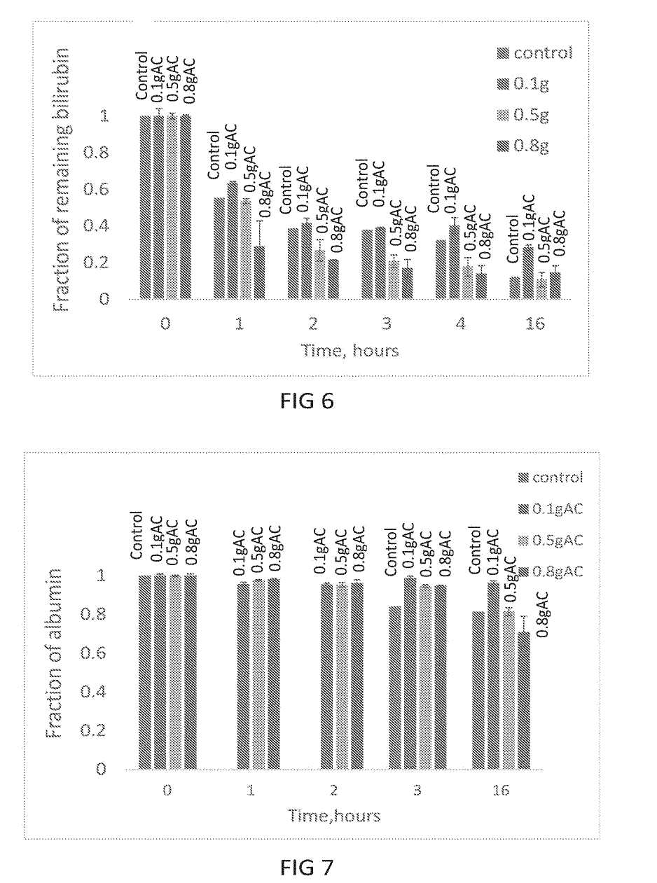

From FIGS. 4 and 5, in albumin free solutions, both control and test, bilirubin showed a decrease in the reading without and with the date pit-derived activated carbon 22 (416 and 350 nm), and the data was not consistent. This decrease in the reading showed the instability of the bilirubin and not because of the adsorption by the date pit-derived activated carbon 22. Bilirubin is an albumin bound toxin and albumin act as a standard clinical marker for liver failure patients. Referring to FIGS. 6 and 7, albumin bound bilirubin was used and it was found that there was a decrease in the reading of bilirubin only in the presence of the date pit-derived activated carbon 22. The control which contain albumin bound bilirubin (PBS) remain stable throughout and from the reading of albumin at 350 nm remained almost similar. Hence the affinity of albumin towards carbon was ruled out.

Scanning electron microscopy is considered as primary tool for characterizing the surface morphology and fundamental physical properties of adsorbent. According to SEM micrograph from FIGS. 1A, 1B, 1C, 2A, 2B and 2C show that the raw material before does not have pore size, while after activation shows cavities. The date pit-derived activated carbon 22 shown in FIGS. 2A, 2B and 2C shows high porosity compared to raw date pit. Pore development in an activated carbon is important since the pores act as active sites, playing the main role in adsorption. Pores formed on surface of adsorbent are sites for toxins to be adsorbed onto the adsorbent. FIGS. 3A, 3B and 3C show the high porous nature of activated carbon over albumin bonded toxin. As can be seen from FIGS. 2A, 2B, 2C, 3A, 3B and 3C, the pores produced in the activated carbon 22 have substantially uniform sizes.

The applicants have found that the date pit-derived activated carbon 22 is an effective adsorbent for removal of protein bound toxins. The applicants have also found that bilirubin is better adsorbed on higher amount of the date pit-derived activated carbon 22.

The inventors envisage that the date pit-derived activated carbon 22 may also be used in other applications other than the treatment described hereinabove. For example, the inventors envisage that the date pit-derived activated carbon 22 may be used for removing other toxins other than those mentioned hereinabove from the body or from other bodily fluids.

The invention extends to the apparatus 10 including the date pit-derived activated carbon 22.

In another embodiment (not shown), the inventors envisage that a permeable membrane made of composite material (not shown) and including the date pit-derived activated carbon 22 can be constructed. The inventors envisage that the permeable membrane can then be placed in a tank containing blood. The inventors envisage that the blood can be purified by a process of diffusion and adsorption through the permeable membrane.

The invention extends also to the use of date pit-derived activated carbon in the manufacture of a pharmaceutical composition and/or permeable membrane for treating biological fluid, for removing protein bound impurities from the biological fluid.

In yet another embodiment (not shown), the inventors envisage that the date pit-derived activated carbon 22 can alternatively be suspended in a tank containing the blood.

The Applicant has advantageously found, from experiments set our more fully below, that the date pit-derived activated carbon 22 is biocompatible with normal liver cells and accordingly suitable for treating biological fluids, such as blood, for purifying the blood. More particularly, the Applicant has advantageously confirmed, by means of experiments set our more fully below, the biocompatibility of the date pit-derived activated carbon 22. More specifically, the Applicant has advantageously confirmed, by means of experiments set our more fully below, that cell viability of 96%-98% was maintained in normal liver cells for up to three hours.

The Applicant has also advantageously found, from experimentation set out more fully hereinbelow, that date pit-derived activated carbon is an effective absorbent for removal of albumin-bound bilirubin, when compared to activated carbon derived from other sources.

The Applicant has furthermore advantageously compared, in experiments set out more fully hereinbelow, the effectiveness of date pit-derived activated carbon to that of activated carbon produced from Jojoba and microalgae (Scenedesmus sp.), and demonstrates that date pit-derived activated carbon 22 has a better adsorption performance and highest bilirubin adsorption with date pit-derived activated concentration of 0.8 g.

The experiments set our hereinbelow provide further examples of the invention and the invention will be further understood with reference to the following experiments which also serve as non-limiting examples of alternative methods of producing date pit derived activated carbon. The particular examples, experiments, materials, amounts and procedures are not intended to limit the scope of the invention.

### EXPERIMENTS

### Materials and Methods

Preparation of Activated Carbon

Date pits were washed with hot water, followed by deionized (DI) water to remove soluble impurities. The washed date pits were then dried in an oven at 100° C. for 2 hours. The dried materials were grinded using an electric agitated mortar (JK-G-250B2, Shanghi Jingke Scientific Instrument), and then sieved using U.S.A standard testing sieve, ASTME-II specification for the size range of 500-600 μm. Carbonization and physical activation were performed in a tube furnace (GSL-1500X, U.S.A). Nitrogen was passed for 10 min, then the temperature was gradually increased from 5° C., under the constant flow of nitrogen, at a rate of 5° C./min to 600° C. and maintained at this temperature for 4 hours. The carbonaceous material was then activated at 900° C. in the same furnace under the flow of carbon dioxide instead of nitrogen, thereby to produce date pit-derived activated carbon (also referred to as “DP-AC”). It will be appreciated that the date pit-derived activated carbon (DP-AC) is the same and/or similar to the date pit-derived activated carbon 22 described hereinabove, and has the same and/or similar properties. It will also be understood that the method 12 may include providing date pit derived activated carbon in accordance with the method described hereinabove.

Similar procedures for producing activated carbon were executed for Jojoba, obtained from Mechanical Engineering Department, at UAEU and Scenedesmus sp. microalgae cells, obtained from Algal Oil Limited, Philippines cells of microalgae. For the production of date pit-derived activated carbon nanomaterials (Nano DP-AC), the date pit-derived activated carbon was wet grinded in Retsch RM 100 grinder and kept frozen dried until used. The date pit-derived activated carbon nanomaterials (Nano DP-AC), will have a particle size of about 60-200 nano meter. It will be appreciated that the Nano DP-AC is the same and/or similar to the date pit-derived activated carbon described hereinabove, and has the same and/or similar properties.

Proximate Analysis:

Proximate analyses of moisture, volatile matter and ash contents were determined for the three biomasses used in this work, according to the ASTM D 121 method and the results are presented in FIG. 9.

Volatile matter content was determined by placing a known quantity of sample in a closed crucible of known dry weight. The crucible was then heated in a muffle furnace (GSL-1500X, U.S.A), and kept at 925° C. for 7.5 min. The crucible was then cooled in a desiccator and the weight of the left over was determined. The volatile matter was determined from the difference in the weights of the sample before and after the heating.

Ash content was determined by placing 1 gm of sample in a silica crucible of known dry weight. The uncovered crucible was heated in the muffle furnace, and kept at 750° C. for 1.5 hr. After that, the crucible was cooled in a desiccator and the weight of the left over was determined. The ash content was determined from the difference in the weights of the sample before and after the heating.

Moisture content: was determined by spreading a known weight of biomass in a petri dish of known dry weight. The uncovered dish was then heated in an oven at a temperature of 105-110° C. for 1.5 hr. After that, the dish was cooled in a desiccator and the weight of the left over was determined. The moisture content was determined from the difference in the weights of the sample before and after the heating.

Adsorption Experiments

Adsorption effectiveness of the date pit-derived activated carbon were compared to that activated carbon (AC) derived from Jojoba and microalgae. Albumin (MW=66000 g mol⁻¹), bilirubin (MW=584.7 g mol⁻¹) and all other chemicals were purchased from Sigma-Aldrich and used as received. To avoid photo degradation of toxins, all experiments were conducted in a dark room and using brown flasks. The stability of the prepared solutions was tested by running control experiments without adsorbents for one week. Bilirubin stock solution of 80 μM was prepared by dissolving 30.4 g of solid bilirubin in 650 mL of 0.1 M NaOH solution. To that, 26 ml of 2% (w/v) albumin solution was added. The volume was completed to 1 L by adding phosphate buffered saline solution, bringing the final pH to 7.4. From the stock solution two dilutions of 60 and 30 μM were prepared.

Batch adsorption experiments were performed in 100 mL dark brown reagent bottle, wherein 40 ml of bilirubin-albumin solutions were mixed with specific amounts of date pit-derived activated carbon, namely 0 g (control), 0.1 g, 0.5 g, and 0.8 g. The bottles were then kept in water bath shaker (Scichemtech, Japan) maintained at a temperature 37° C., to mimic the human body temperature. The shaking speed was kept constant for all the runs, which was high enough to uniformly disperse the date pit-derived activated carbon in the solution. For comparison, the same experiment was repeated using AC prepared from Jojoba and microalgae.

Analysis of albumin-bilirubin solutions was carried out spectrophotometrically by a UV-visible spectrophotometer (Shimadzu UV 1800, Japan). Total bilirubin concentration was measured at a wavelength of 416 nm, at which the bilirubin extinction coefficient does not depend on albumin/bilirubin molar ratio, and albumin concentration was measured at a wavelength of 279 nm. The calibration was obtained by measuring the optical density of known concentrations of bilirubin and albumin at their respective wavelengths.

Date Pit-Derived Activated Carbon Characterization:

Surface morphology of raw date pits and the date pit-derived activated carbon were performed using 3 KV accelerating voltage Scanning electron microscopy (JSM-5600, Jeol Ltd.). The samples were dried overnight at approximately 105° C. before SEM analysis. Oven dried, samples were mounted on an adhesive carbon tape attached to an aluminum-stub and subsequently sputter coated with gold layer and the samples were then analyzed using scanning electron microscope.

Chemical characterization was carried out by Fourier Transform Infrared (FTIR) spectroscopy (Nexus 470 FTIR Spectrophotometer) to determine the variations in the functional groups at the surface of the date pit-derived activated carbon. For this analysis, date pits and date pit-derived activated carbon were oven dried at 110° C. overnight, stored in capped flasks and kept in a desiccator prior analysis. Test samples were prepared by uniformly dispersing the particles in KBr and compressing them into pellets. A sample of 10 mm diameter was prepared by taking a small amount of powder sample (about of 0.1-2% of the KBr amount, or just enough to cover the tip of spatula) and mixed with the KBr powder, subsequently the mixture was grinded for 3-5 minutes to form a pellet. Then the pellet was placed in sample holder and the spectrum was recorded in the wavenumber range 4000-400 cm⁻¹.

Thermogravimetric Analysis (TGA) was used to measure the amount and the rate of weight change of the date pit-derived activated carbon as a function of temperature in a controlled environment. Approximately 12 mg of DP-AC was placed in a platinum crucible (Q-50, TA Instruments) on the pan of a microbalance and then heated between 25° C. to 800° C. at a heating rate of 10° C. min⁻¹ with nitrogen flow rate 40 mL min⁻¹.

date pit-derived activated carbon melting point and glass transition temperature were also examined using a Differential Scanning Calorimetry (DSC). A sample of 5 mg was heated from 25 to 600° C., at a heating rate of 10° C. min⁻¹, with a nitrogen flow rate of 50 mL min⁻¹. Heat flow (w/g) vs temperature (° C.) plots were taken by Q-200, TA Instrument.

Cytotoxicity

The biocompatibility of the prepared date pit-derived activated carbon was studied by determining its cytotoxicity on normal liver cells to determine whether the date pit-derived activated carbon is suitable for use in removal of blood protein bound toxins, without having a toxic effect on the patient. For this, THLE-2 cells were used and cytotoxicity assay, namely MTT, was conducted. The toxic effects were analyzed at three different time points, 1^{st} hour, 2^{nd} hour and 3^{rd} hour.

THLE-2 cells, purchased from the American Type Culture Collection (ATCC; Rockville, Md., USA), were maintained in the Bronchial Epithelial Cell growth medium (BEGM, Lonza) supplemented with 10% FBS. Prior experiments, frozen medium was removed by suspending the contents of one vial in 10 ml of propagation medium in a 50 mL falcon tube. Thawed cells were centrifuged at 1,000 rpm for 5 min at 4° C. The cell pellets were re-suspended in propagation medium and were seeded in T-25 flasks pre-coated with collagen I (2.9 mg mL⁻¹), fibronectin (1 mg mL⁻¹) and bovine serum albumin (1 mg mL⁻¹) in bronchial epithelial growth medium according to manufacturer guidelines. The cells were incubated at 37° C. and 5% CO₂ for 2-3 days till they attained 40-60% confluency. The cells were then dissociated using trypsin-EDTA solution, re-suspended in their respective medium and counted using a hemacytometer. Cell viability assay was done using the Trypan blue exclusion assay method and was determined to be over 90% prior to the seeding of cells.

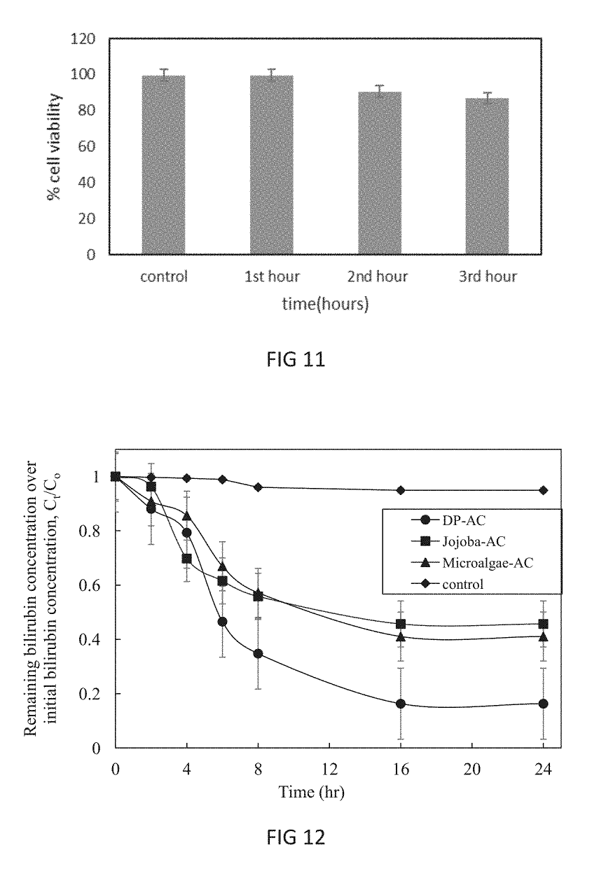

For MTT toxicity, THLE2 cells were seeded at 5×10³ cells mL⁻¹ by adding 100 μL of the cell suspension to each well of a 96-well tissue culture plate. The medium was aspirated off and replaced with fresh medium (100 μL) containing nanomaterials of date pit-derived activated carbon, i.e, Nano DP-AC. Untreated cells culture was considered as a control and the medium alone as a blank. The plates were incubated at 37° C., 5% CO₂, for one hour, two hours and three hours. After incubation, the medium was aspirated off and replaced with fresh medium. Then, MTT solution (10 μL) for a total volume of 100 μL was added in every well and incubated for 3 hours at 37° C. with 5% CO₂. After that, MTT-containing medium was removed gently, replaced with DMSO (100 μL per well) to mix the formazan crystals until dissolved. After 20 minutes the plates were read on microtiter plate reader at 570 nm. FIG. 11 show the results of the cytotoxicity tests. In FIG. 11, the viability percentage was expressed as absorbance in the presence of test compound as a percentage of that in the vehicle control.

Immunohistochemistry (H&E Staining)

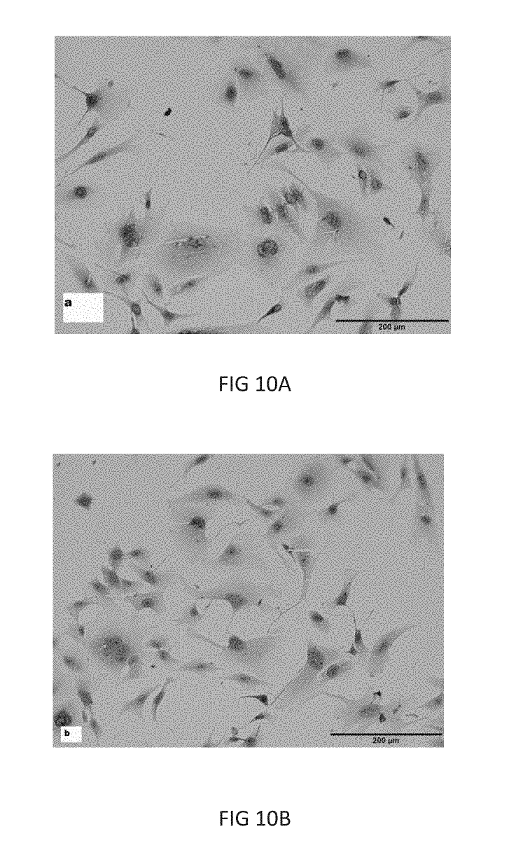

Cultured THLE-2 cells with the carbon nanomaterial were washed with phosphate buffered saline (PBS), fixed with ice cold methanol:ethanol (1:1 ratio) for 15 minutes and air dried. The coverslips were washed quickly 5-6 times in distilled water. The cells were stained with haematoxylin solution for 1-2 minutes and then rinse in tap water. Counter stained with eosin for 10 seconds. The coverslips were rinsed in ethanol series (70%, 96%, and 100%). Air dried and coverslips were mounted with DPX and examined under bright field microscope. The results are shown in FIGS. 10A and 10B.

Statistical Analysis

Each experiment was repeated three time, and the average values of the results were then presented. The reproducibility of the experimental results was evaluated using the standard deviations and shown as error bars (mean+−SD) in FIGS. 11 to 14 for adsorption tests and cytotoxicity experiments. The differences between the groups were analyzed using t-test post hoc analysis, and a p-value that was lower than 0.05 was considered statistically significant.

Results and Discussions

### Bilirubin Adsorption on Date Pit-Derived Activated Carbon

Albumin binds strongly to water-insoluble bilirubin, which makes it difficult to remove using convectional hemodialysis method. An increase in bilirubin levels could cause severe motor symptoms, cerebral palsy and renal dysfunctions. Therefore, an effective and reliable method for bilirubin removal is required. The effectiveness of bilirubin removal by adsorption was tested in the presence of albumin.

The bilirubin adsorption using 0.8 gm of activated carbon from different sources, namely date pits, Jojoba and microalgae, at an initial bilirubin concentration of 30 μM, is shown in FIG. 12. The data shown in FIG. 12 are the average of multiple repetitions of the experiment, with the standard deviation shown as error bars. The lines are connections between the experimental results shown to highlight the trend. The insignificant drop in the bilirubin concentration in the control experiments (p<0.05), proves that the drop was mainly due to the adsorption of the bilirubin. The performance of date pit-derived activated carbon was superior to that of the other two adsorbents, in both adsorption rate and capacity. This may be due to high number of active sites present in the date pit-derived activated carbon. The highest adsorption rate of bilirubin was observed within the first 4 hours. The adsorption usually goes through two stages. In the initial stage, external surface adsorption dominates, where the bilirubin compounds diffuse from the solution to the external surface of the adsorbent, which is mainly controlled by the external boundary layer. The second stage starts when the external surface is almost totally saturated with the bilirubin molecules, and internal diffusion becomes more important, which results in a gradual decrease in the adsorption rate.

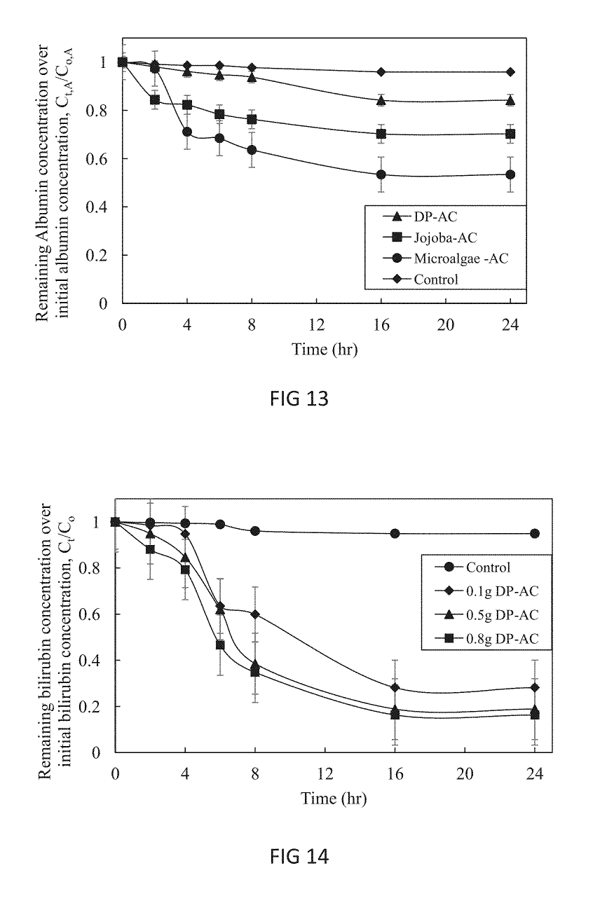

The albumin concentration was also monitored throughout the experiment, and it was interesting to notice that in the presence of date pit-derived activated carbon (DR-AC), the drop in the albumin concentration was insignificant, as shown in FIG. 13. However, with other adsorbents, the drop in the albumin concentration was more significant. This suggests that the date pit-derived activated carbon was able to strip off the bilirubin molecules from the bilirubin-albumin complex, leaving the albumin behind, rather than adsorbing the complex altogether. This indicates that the date pit-derived activated carbon is appropriate for the removal of bilirubin from the albumin bound bilirubin solutions, which shows its high adsorbent property. Albumin concentration remained constant. Similar to FIG. 12, the data shown in the FIG. 13 are the average of multiple repetitions of the experiment, with the standard deviation shown as error bars. The lines are connections between the experimental results shown to highlight the trend.

Further adsorption tests were carried out using different amounts of date pit-derived activated carbon, 0.1, 0.5 and 0.8 g and different bilirubin initial concentrations, 30, 60, 80 μM. The results at initial concentration of 30 μM, in FIG. 14, show that the adsorbed bilirubin amount increased with the increase in amount of adsorbent. This was expected because with the increase in the adsorbent amount, the available adsorption sites increase. Similar results were also observed at the other initial bilirubin concentrations. The data shown in FIG. 14 are the average of multiple repetitions of the experiment, with the standard deviation shown as error bars. The lines are connections between the experimental results shown to highlight the trend.

Surface Morphology by SEM:

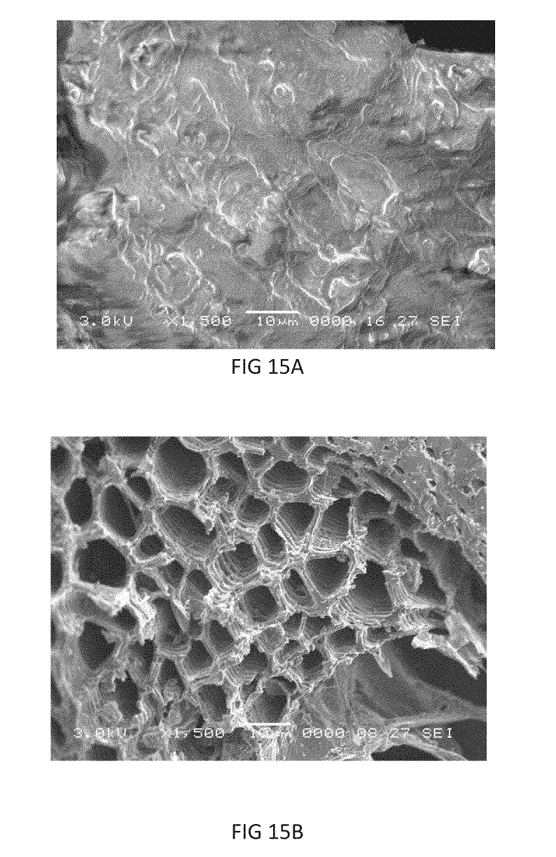

FIGS. 15A and 15B shows the SEM images, at ×1500 magnification, of the date pits before and after activation, respectively. It was noticed that the porosity of date pit-derived activated carbon significantly increased compared to the raw date pits. This is because the pores were created during the carbonization step due to the escape of the volatile constituents in the date pits and the thermal degradation of some macromolecules.

Chemical Analysis by FTIR

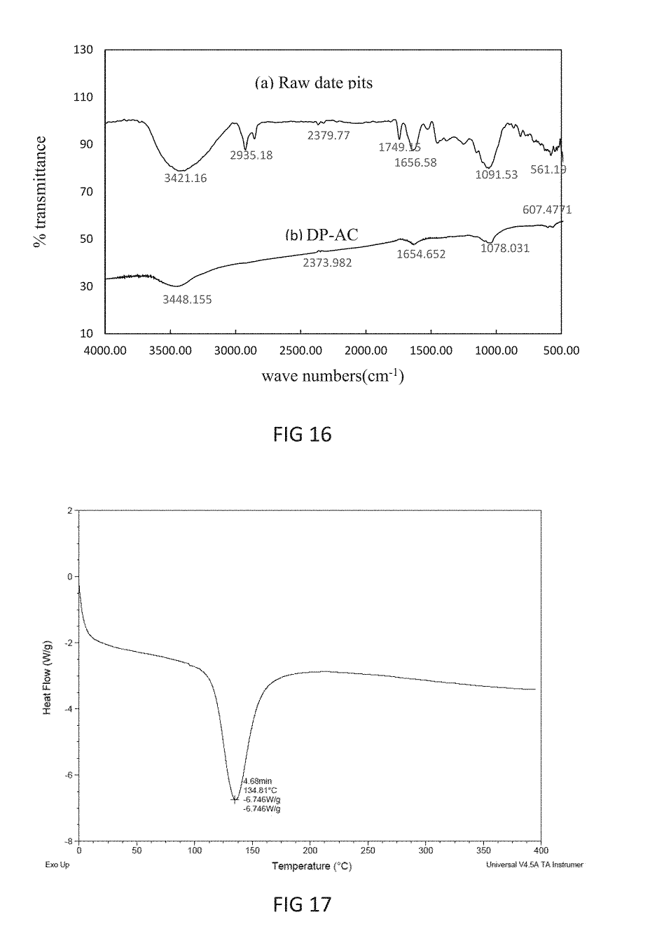

FTIR spectroscopy, a chemical characterization technique, was used to identify the functional groups in the raw date pits and the date pit-derived activated carbon. The spectrum in an FTIR graph consist of two regions, fingerprint region at wave numbers 650 to 1400 cm⁻¹ and a functional group region for wave numbers from 1400 to 4000 cm⁻¹. An IR band at about 600 cm⁻¹ attributes to in-plane ring deformation. Bands at about 1000 cm⁻¹ and 1500 cm⁻¹ correspond to C—O stretching in acids, alcohols, phenols, ethers and esters and C═O stretching of lactonic and carbonyl groups, respectively. Bands at around 2900 cm⁻¹ and 3500 cm⁻¹ are due to C—H and O—H stretching, respectively. The spectra of the raw date pit, in FIG. 16, showed significant absorption peaks. For date pit-derived activated carbon, the peak for C—H at 2900 cm⁻¹ completely disappeared, whereas for O—H, C—O and C═O, the peaks intensities dropped significantly. This change in intensity is a result of the activating action which caused O₂ and H groups to decompose. The surface functional groups on date pit-derived activated carbon can be determined using the Boehm titration method.

Differential Scanning Calorimetery DSC:

FIG. 17 shows the pictorial representation of the DSC curve of date pit-derived activated carbon. The glass transition temperature for this material occurred at ≈85° C. Additionally, the melting temperature was represented by the endothermic peak at ≈135° C. At heating rate of 10° C. min′, maximum slope, peak and end of second endothermic peak (solids-melting) were observed at 100, 135 and 160° C., respectively. These structural characteristics determined using the DSC shows the naturally heterogeneity of date-pits with multicomponent mixture.

Thermo Gravimetric Analysis (TGA)

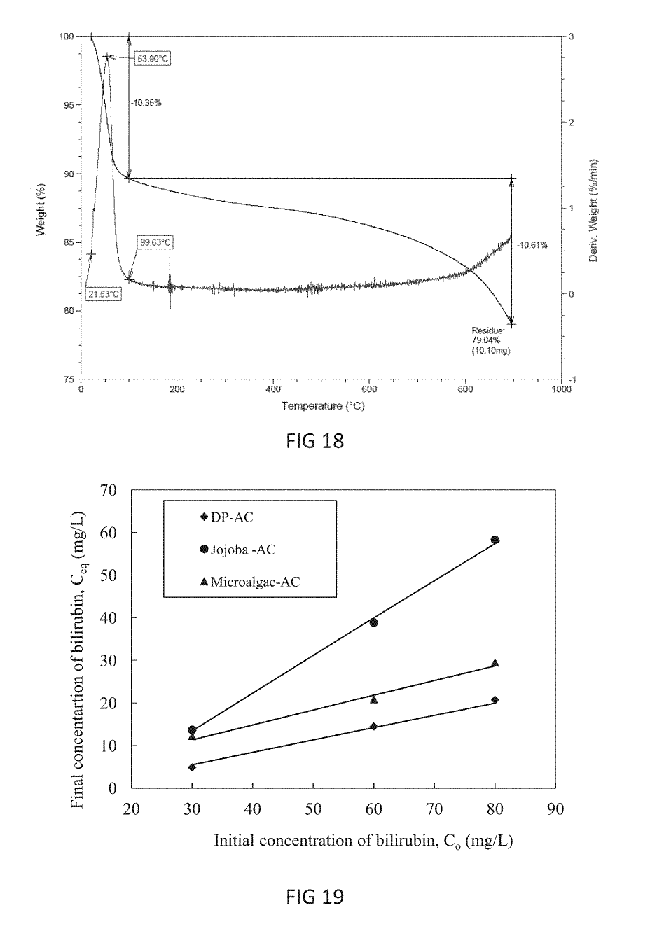

FIG. 18 shows the Thermo-gravimetric analysis (TGA) and differential thermogravimetric analysis (DTG) profiles of the date pit-derived activated carbon, which gives an approximation about the weight loss of the sample with temperature. The first decomposition stage occurred between 21.53° C. up to ≈99.63° C. where a steep weight loss of 10.35% was observed. This can be attributed to the release of surface bounded water and volatile matters. The highest rate of degradation in this stage was indicated by the peak on the DTG curve at a maximum rate of decomposition temperature of 53.9° C. The second stage showed a slower rate of weight loss with only 10.61% change over a wide range of temperature between 100° C. up to 900° C. The weight loss in this stage and specifically until a temperature of ≈450° C. was due to the decomposition of cellulose and hemicellulose constituents. For the higher temperatures above ≈450° C. the weight loss was due to lignin decomposition. The test was terminated at a temperature of 900° C. where the amount of ash remaining was 79.04% which is considered as indication for the thermal stability of the material under investigation (date pits activated carbon) over a broad range of temperatures.

Adsorption Isotherms

The equilibrium attained, between the solute remaining in the solution and that present at the surface, is expressed by adsorption isotherm. FIG. 19 shows the results at different initial concentrations of bilirubin using 0.8 g adsorbents. The data shown in FIG. 19 are the average of multiple repetitions of the experiment, with the standard deviation shown as error bars. The lines are connections between the experimental results shown to highlight the trend. The equilibrium concentration of the solute, C_{eq}, was determined from the amount of bilirubin left after 16 hours contact with the date pit-derived activated carbon, shown in FIG. 19. The amount of solute adsorbed at equilibrium, q_{eq} was then determined using Eq (1).

q_{eq}=(Cₒ−C_{eq})Vₛₐₘₚₗₑ/m (1)

Where, q_{eq} (mg g⁻¹) is the amount of solute adsorbed at equilibrium, Cₒ in the initial bilirubin concentration (mg/L), C_{eq} (mg L⁻¹) is the equilibrium concentration of the solute in the bulk solution after 16 hours, Vₛₐₘₚₗₑ (L) is the volume of the sample and m (g) is the mass of the adsorbent used.

Several models have been suggested to describe this equilibrium relationship. Among these models, Langmuir and Freundlich isotherms are the commonly used. The Langmuir isotherm, described by Eq (2), assumes that forces of attraction between the adsorbed molecules are negligible, and when a molecule occupies one site, then no further adsorption takes place at that site.

q_{eq}=qₒbC_{eq}/(1+bC_{eq}) (2)

Where, qₒ (mg g⁻¹) is the maximum adsorption capacity and b (L mg⁻¹) is constant related to free energy of adsorption. By plotting the graph 1/qₑ vs 1/C_{eq}, Langmuir constants can be determined, and the values of qₒ and b could be determined from the intercept and slope respectively. The results shown in FIG. 19, were used to determine q_{eq} and C_{eq}, which were then used to determine the isotherm parameters qₒ and b.

On the other hand, the Freundlich isotherm, shown in Eq (3) describes the non-ideal and reversible adsorption, applied to multilayer adsorption with non-uniform distribution of adsorption heat and affinity over the heterogeneous surface.

q_{eq}=k_{f}C_{eq}^{1/n} (3)

Where, k_{f} and n are Freundlich parameters, which can be determined from the slope and intercept of the straight line of log (q_{eq}) versus log C_{eq}.

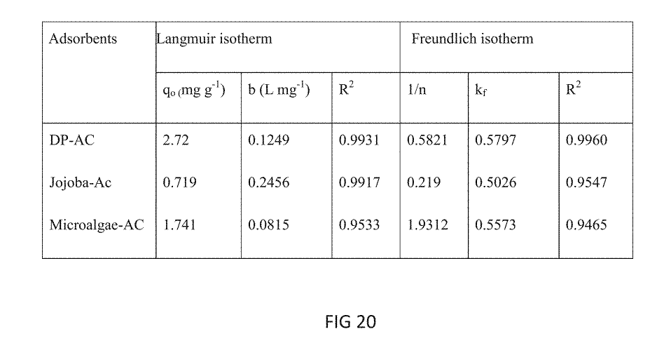

The determined parameters of the two isotherms and their respective coefficient of determination, R², which measures the goodness of the fitting, are presented in FIG. 20. For date pit-derived activated carbon, the R² of the Langmuir and Freundlich fittings was close to unity, which suggests that both models can well describe the adsorption process. However, for Jojoba and microalgae, Langmuir model was more suitable.

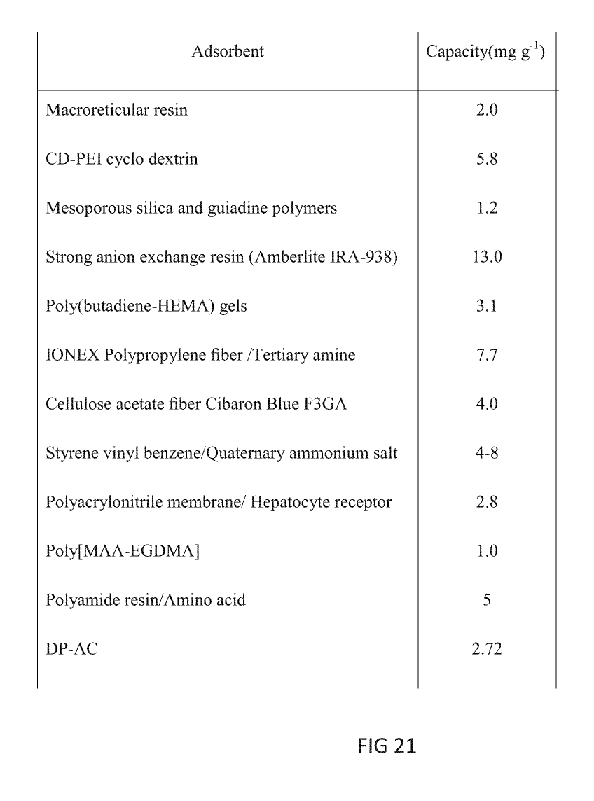

The maximum capacity of date pit-derived activated carbon (DP-AC) was determined to be 2.72 mg g⁻¹, and was higher than Jojoba-AC and microalgae-AC (0.719 and 1.741 mg g⁻¹, respectively), which agrees with the results shown in FIG. 12. A comparison of the bilirubin adsorption capacity of the date pit-derived activated carbon, with those of other adsorbents reported in the literature is presented in the FIG. 21, which shows that the capacity of the date pit-derived activated carbon was comparable to those of other commercially available adsorbents.

Adsorption Kinetics Studies:

If surface adsorption is slow, compared to the internal diffusion, then the process is adsorption controlled. In this case, either first order or second order adoption kinetics models are usually used to describe the process. However, if internal diffusion is slow in comparison to the surface adsorption kinetics, and in this case the system is described by the internal diffusional model. In these experiments, the kinetics experimental results of bilirubin adsorption were fitted to three kinetics models, namely pseudo first order and pseudo second order kinetic model and Intra particle diffusion models, to understand the controlling mechanism of the adsorption processes. The developed kinetics model with the experimentally determined model parameters can be used for the scale-up and design of fixed-bed adsorption columns.

Pseudo First Order Kinetic Model:

The linear form of pseudo first order model can be expressed as in Eq (4)

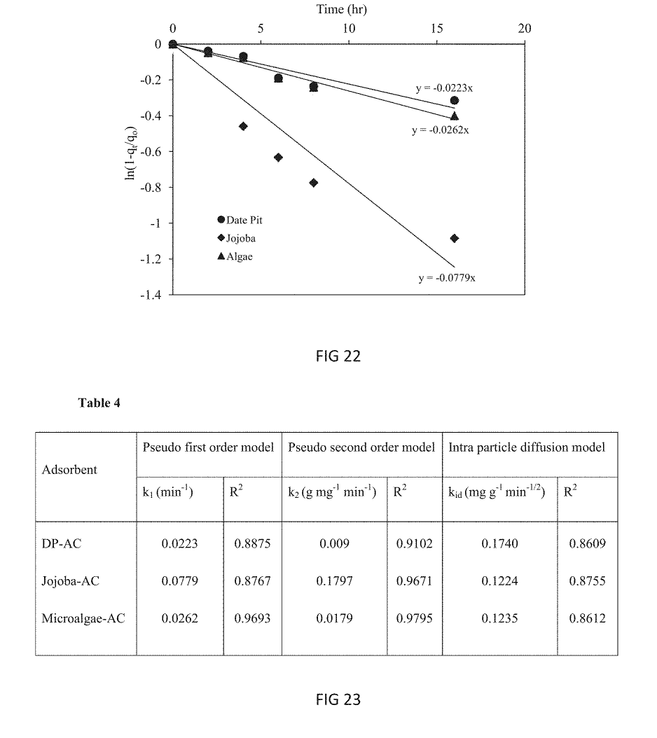

ln [1−(qₜ/q₀)]=−k₁t (4)

Where, qₒ (mg g⁻¹) is the maximum capacity, determined from the Langmuir isotherm and qₜ (mg g⁻¹) is the adsorbed bilirubin at any time, which is determined from Eq (1) but replacing C_{eq} with the concentration of the solute in the bulk solution at any time, Cₜ. The pseudo first-order rate constant, k₁ (min⁻¹), can be determined from the slope of the straight line of ln [1−(qₜ/qₑ)] vs t plot, as shown in FIG. 22. The determined values of k₁ for the three tested adsorbents with their respective coefficient of determination R², are shown in FIG. 23.

### Pseudo Second Order Kinetic Model:

The linear form of pseudo second order model can be expressed as in Eq (5)

$$\frac{1}{{q}_{o} - {q}_{t}} - \frac{1}{{q}_{o}} = {k}_{2} t \qquad ( 5 )$$

Where, k₂ (g mg⁻¹ min⁻¹) is the pseudo second-order rate constant, which can be determined from the slope of the straight line of

$$( \frac{1}{{q}_{o} - {q}_{t}} - \frac{1}{{q}_{o}} )$$

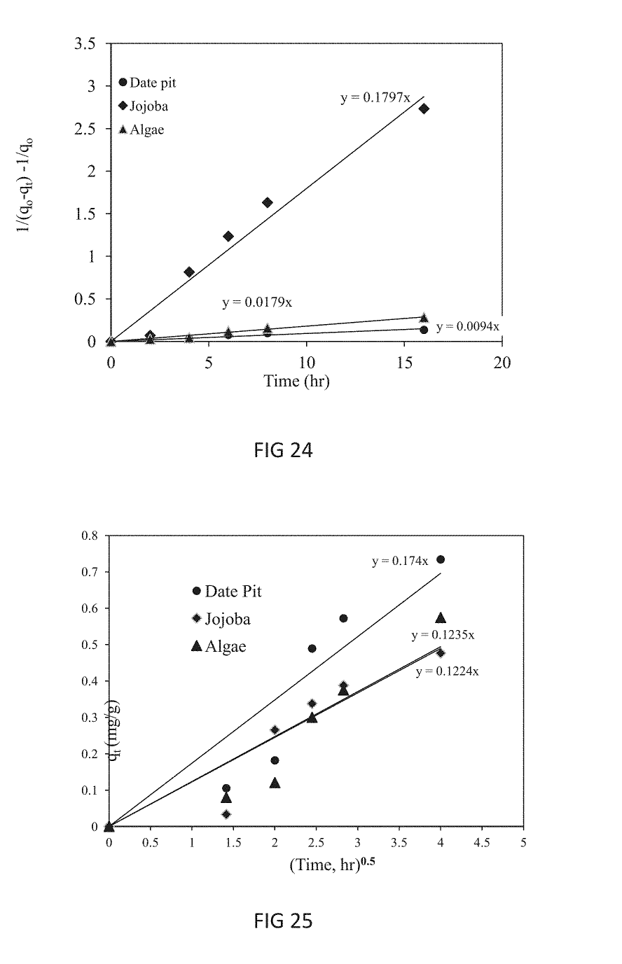

vs t plot, as shown in FIG. 24. The determined values of k₂ for the three tested adsorbents with their respective coefficient of determination R², are shown in FIG. 23.

### Intra Particle Diffusion Model:

According to the intra-particle diffusion model, proposed by Morris, Wu, Feng-Chin, Ru-Ling Tseng, and Ruey-Shin Juang. “Kinetic modeling of liquid-phase adsorption of reactive dyes and metal ions on chitosan.” Water Research 35.3 (2001): 613-618 the adsorbate uptake varies almost proportionally to the square root of the time, as shown in Eq (6)

qₜ=kᵢₛt^{1/2} (6)

Where, k_{id} (g mg⁻¹ min^{-1/2}) is the internal diffusion constant, which can be determined from the slope of the straight line of qₜ vs t^{1/2} plot, as shown in FIG. 25. The determined values of k_{id} for the three tested adsorbents with their respective coefficient of determination R², are shown in FIG. 23. As shown from the results in FIG. 23, the kinetics of the adsorption of bilirubin for all tested adsorbents were best described by the second order, with R² values closest to unity. This indicates that the rate-limiting step is the chemisorption, which further supports the earlier finding that the DP-AC stripped off the bilirubin from the complex, rather than adsorbing the entire complex, which is more likely to have taken place with physical adsorption. The pseudo-second-order model has been widely accepted for the description of adsorption kinetics in liquid-phase adsorption systems. The adsorption kinetics of phenol from wastewater was tested using date pit-derived activated carbon, which was also found to be better described by the pseudo second order model. The adsorption process proceeds through the following sequence of steps: transport of the adsorbate from the boundary film to the external surface of the adsorbent (film diffusion); transfer of adsorbate from the surface to the intraparticular active sites; and uptake of adsorbate by the active sites of adsorbent. The results show that the intraparticle diffusion was not controlling, which was expected due to the large pore sizes, as shown in FIG. 15B.

The driving force for adsorption is the difference between the capacity at any time and the maximum capacity of the adsorbent. Therefore, date pit-derived activated carbon has the highest driving force, due to its high maximum capacity, compared to the other tested adsorbents, as determined from the adsorption isotherm studies. Due to this high driving force, the kinetics constants of date pit-derived activated carbon were found to be lower than those of the other two adsorbents. The determined pseudo second order kinetics model can now be used for the scale-up and design of fixed-bed adsorption columns for removing of bilirubin from blood stream of liver failure patients.

### Cytotoxicity of Nano Materials on THLE2 Cells

The viability of the normal liver cells, namely THLE2 cells, was used to assess the toxicity of the produced date pit-derived activated carbon using the MTT assay. Up to 3 hours of hemodialysis has been suggested as a method of removing dabigatran and thereby reducing its anticoagulant effect. The THLE2 cells were co-cultured with nano-materials derived from date pit-derived activated carbon (Nano-DP-AC) for three hours at 5 μL mL⁻¹, of Nano-DP-AC concentration. As show in FIG. 11, cytotoxic activity was not observed in the cells when incubated with the Nano-DP-AC in the first two hours. Only a marginal activity was observed during these times, and therefore the cell viability percentage was not affected muchand remained almost same as the control (p>0.05). A slight cytotoxicity (p<0.05) was observed in the THLE2 cells when incubated with Nano-AC-DP for three hours, with 84% viability, compared to the control with 96% viability). Nevertheless, this drop was not significant, and could be avoided in real liver failure treatments, by limiting the adsorption process to less than 2 hours. In addition, the cytotoxic activity was not significant as the concentrations of Nano-DP-AC increased by five folds to 25 μL mL⁻¹ (results are not shown),

### Cells Morphology

To further confirm the biocompatibility of the produced date pit-derived activated carbon, the morphology of exposed THLE2 cells to Nano-DP-AC for 3 hours was analyzed using inverted fluorescence microscope, and compared to the morphology of unexposed cells (Control). No differences in cells morphology was observed, as shown in FIGS. 10A and 10B of the drawings, which confirms the biocompatibility of the produced adsorbent.

### CONCLUSION

Activated carbon produced from date pits was shown to be an effective adsorbent of bilirubin from solutions containing bilirubin-albumin complex, while leaving the albumin intact. The capacity of the date pit-derived activated carbon was comparable to other adsorbents found in literature, and was found to be higher than those of AC produced from Jojoba and microalgae. The experimental results were used to determine the parameters of equilibrium isotherms and kinetics models. The results show that the process was better described by the Langmuir isotherm and the pseudo second order kinetics model. The cytotoxicity of the produced date pit-derived activated carbon was tested against normal liver cells. It was shown that cells viability of 98-96% was maintained for up to 3 hours, which confirms the biocompatibility of the adsorbent.

The Applicant believes that the date pit-derived activated carbon 22 and the method 12 is particularly advantageous as the date pit derived activated carbon 22 is effective in removing bilirubin from the blood. Furthermore, the Applicant believes that the date pit derived activated carbon 22 and the method 12 is particularly advantageous as the date pit-derived activated carbon 22 is cheap to produce, when compared to activated carbon produced from other sources, as date pits are cheap to obtain because date pits are a waste product of commercially produced dates.

While the present invention has been described with respect to specific examples, it should be appreciated that the present invention is not limited to these examples. It is to be believed that one skilled in art, using the preceding description, can utilize the present invention to its fullest extent, and many variations and modifications may present themselves to those of skill in the art without diverting from the scope of the present invention.

1. A method for treating a biological fluid obtained from a patient, for detoxifying the biological fluid, the method comprising:
providing date pit-derived activated carbon; and
contacting the biological fluid with the date pit-derived activated carbon.

2. The method for treating as claimed in claim 1, wherein the method for treating is a method for detoxifying the biological fluid by removing protein bound impurities from the biological fluid.

3. The method for treating as claimed in claim 2, wherein the contacting of the biological fluid with the date pit-derived activated carbon results in sorption of the protein bound impurities present in the biological fluid, by the date pit-derived activated carbon.

4. The method for treating as claimed in claim 1, wherein the contacting of the biological fluid with the date pit-derived activated carbon comprises soaking the date pit-derived activated carbon within the biological fluid.

5. The method for treating as claimed in claim 4, wherein the date pit-derived activated carbon is soaked within the biological fluid for a period of time of approximately 10-16 hours.

6. The method for treating as claimed in claim 1, wherein the method includes grinding the date pit-derived activated carbon so as to obtain granules of the date pit-derived activated carbon.

7. The method for treating as claimed in claim 1, wherein the method further includes controlling a pH of the biological fluid so as to maintain the pH at approximately pH 6.9-7.9, preferably pH 7.4.

8. The method for treating as claimed in claim 1, wherein the method further includes controlling a temperature of the biological fluid so as to maintain the temperature at approximately 36.9-37.9° C. preferably approximately 37° C.

9. The method for treating as claimed in claim 1, wherein the method includes obtaining the biological fluid from the patient.

10. The method for treating as claimed in claim 1, wherein the method includes treating batches of the biological fluid, whereby a volume of the biological fluid is obtained from the patient and thereafter, the treated biological fluid is returned to the patient.

11. The method for treating as claimed in claim 1, wherein the biological fluid is in the form of blood and wherein the method is for the removal of protein bound impurities from the patient's blood.

12. The method for treating as claimed in claim 11, wherein the protein bound impurities removed from the blood are in the form of albumin bound toxins.

13. The method for treating as claimed in claim 11, wherein the protein bound impurities removed from the blood are in the form of bilirubin.

14. The method for treating as claimed in claim 1, wherein providing date pit-derived activated carbon includes providing date pits and physically and chemically activating the date pits so as to obtain said date pit-derived activated carbon.

15. The method for treating as claimed in claim 14, wherein the physical activation includes carbonizing the date pits in an inert atmosphere.

16. The method for treating as claimed in claim 15 wherein the physical activation further includes exposing the carbonized date pits at an elevated temperature to at least one of oxidizing gasses and steam.

17. The method for treating as claimed in claim 15 wherein the chemical activation includes exposing the carbonized date pits to dehydrating chemicals.
